(19) United States
(12) Patent Application Publication
(10) Pub. No.: US 2022/0031496 A1
(43) Pub. Date: Feb. 3, 2022

(54) CONDOM HAVING INSTRUCTIONAL INDICIA

(71) Applicant: Philip T. Jenkins, Cliffside Park, NJ (US)

(72) Inventor: Philip T. Jenkins, Cliffside Park, NJ (US)

(21) Appl. No.: 16/944,754

(22) Filed: Jul. 31, 2020

(51) Int. Cl. A61F 6/04

(52) U.S. Cl. CPC A61F 6/04; A61F 6/065

(57) ABSTRACT

A condom is disclosed, comprising a tubular body having an open end and a closed end, the tubular body comprising an inner surface and an outer surface, and the tubular body provided in a rolled configuration. A first texture is provided on the inner surface to give a first indication of the orientation of the tubular body. A second texture is provided on the outer surface the second texture provides a second indication of the orientation of the tubular body. At least one indicator is provided on the inner surface or the outer surface to provide an indication of the orientation of the tubular body.

### TECHNICAL FIELD

The embodiments generally relate to condoms and, more specifically, relate to multi-toned and multi-textured condoms to instruct the user how to properly wear the condom.

### BACKGROUND

Condoms are well known prophylactic devices. The condom is a sheath-shaped barrier device used during sexual intercourse to reduce the probability of pregnancy and/or contracting a sexually transmitted infection (STI). Often, condoms are constructed of natural latex, synthetics, or lambskin. Using a condom reduces the probability of becoming pregnant by up to 98% and result in a 99.9% reduction in the likelihood of contracting gonorrhea, chlamydia, trichomoniasis, hepatitis B, and human immunodeficiency virus (HIV). They also protect, to a lesser extent, against genital herpes, human papillomavirus (HPV), and syphilis. In order to work effectively, the condom must be worn correctly to form a sufficient physical barrier. Condoms are packaged in foil or plastic in a rolled-up configuration. The condom is rolled onto an erect penis before intercourse to form the physical barrier.

Many condoms that are currently available include textures on at least one side of the sheath to stimulate the user. Further, some condoms include coloring for aesthetic purposes. Current prophylactics often lack instructions for their proper use.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide a condom comprising a tubular body having an open end and a closed end, the tubular body comprising an inner surface and an outer surface, and wherein the tubular body is provided in a rolled configuration. A first texture is provided on the inner surface to give a first indication of the orientation of the tubular body. A second texture is provided on the outer surface, the second texture provides a second indication of the orientation of the tubular body. At least one indicator is provided on the inner surface or the outer surface to provide an indication of the orientation of the tubular body.

The condom provides various indicia including textures, colors, and indicators to instruct the user on how to properly wear the condom. The condom is constructed to provide a physical barrier to reduce the probability of contracting a sexually transmitted infection. Further, the condom reduces the likelihood of the sexual encounter resulting in a pregnancy.

In one aspect, a first color is provided on the inner surface. The first color is used to indicate the inner surface and direct the user to the correct use of the condom.

In one aspect, a second color provided on the outer surface. Similar to the first color, the second color is used to indicate the outer surface and direct the user to the correct use of the condom.

In one aspect, the indicator is a symbol oriented to indicate the closed end of the condom.

In another aspect, the indicator is a symbol oriented to indicate the open end of the condom.

In one aspect, the second color is visible as an annular ring at a bottom end of the condom when provided in the rolled configuration.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of the condom in a rolled configuration, according to some embodiments;

FIG. 2 illustrates a perspective view of the condom in an in-use and unrolled configuration, according to some embodiments; and

FIG. 3 illustrates a cross-section view of the condom showing the inner surface thereof, wherein the condom is in an in-use and unrolled configuration, according to some embodiments.

### DETAILED DESCRIPTION

The specific details of the single embodiment or variety of embodiments described herein are to the described apparatus. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The specific details of the single embodiment or variety of embodiments described herein are set forth in this application. Any specific details of the embodiments are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

In general, the embodiments provided herein relate to a condom having a plurality of characteristics which direct the user to wear and use the condom properly. The condom may include one or more colors corresponding to a surface of the condom. For example, the condom may include an outer surface having a first color and an inner surface having a second color. In such, the user is provided with a color indication of the proper orientation of the condom when it is applied onto the user. Similarly, the condom may include two or more textures corresponding to a surface of the condom. For example, the condom may have a first texture on the outer surface and a second texture on the inner surface to direct the user to the appropriate method and position of wearing the condom when applied onto the user. An indicator may be provided on the inner and/or outer surface to provide an indicator for the correct orientation of applying and wearing the condom.

As used herein the term “indicia” may be used to describe the texture(s), color(s), or indicator(s) provided on the condom. One skilled in the arts will readily understand that any combination of indicia may be used to instruct the user to properly wear the condom. For example, the condom does not necessarily require two textures, two colors, and two indicators and may only include a subset of these. In one example, a first texture, first color, and first indicator are each provided on the outer surface of the condom as described in some embodiments herein.

As used herein, the term “condom” may refer to male condoms and/or female condoms. It is a goal of the embodiments to provide a male or female condom having various colors, textures, and symbols (indicia) to instruct the male and/or female user of the appropriate and correct application and use of the condom.

FIG. 1 illustrates a perspective view of the condom 100 configured as a roll 102. The condom 100 is provided to the user in a rolled configuration wherein the roll 102 may be packaged in foil, plastic, and other materials. One skilled in the arts will readily understand that the packaging may be provided in various methods known in the arts. During production, the condom 100 is rolled such that the inner surface 104 forms an annular ring 106 around the open end 108 of the condom 100. The closed end 110 is positioned at a top end 112 of the condom and which is not rolled to allow the user to properly position the condom 100 on the anatomy and begin unrolling the condom 100. The outer surface 114 may include first indicator 116 to indicate the position in which the condom 100 is oriented.

In some embodiments, the first indicator 116 is positioned at the top end 112 of the condom 100. The first indicator 116 may comprise various symbols to indicate the surface of the condom 100 on which it is positioned. As shown in the illustrated example, in FIG. 1, the first indicator 116 is a female symbol, indicating that it is positioned on the outer surface 114, which contacts the female during use.

In some embodiments, the symbol may include imagery, text, characters, and the like. The symbol may be embossed, texturized, etc. to further indicate the correct application and use of the condom.

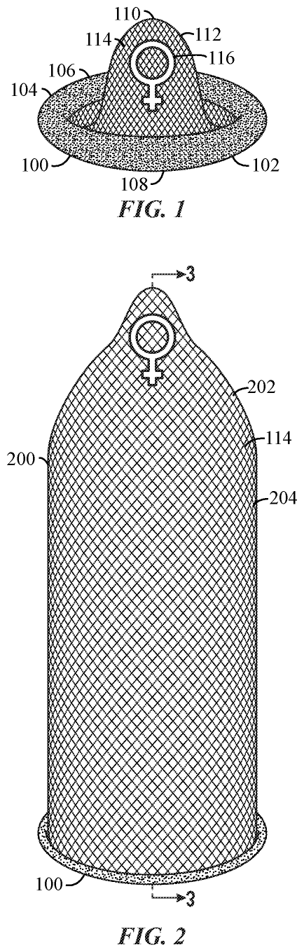

FIG. 2 illustrates the condom 100 in an unrolled and in-use configuration wherein the condom 100 has been unrolled to expose the tubular body 200 of the condom 100. The tubular body 200 is provided with a first texture 202 forming at least a portion of the outer surface 114 of the condom 100. The first texture 202 may be provided on the only a portion of the outer surface 114 or may be present on the entire outer surface 114. The outer surface 114 may include a first color 204, which may be used to further indicate the appropriate orientation of the condom 100 when it is applied.

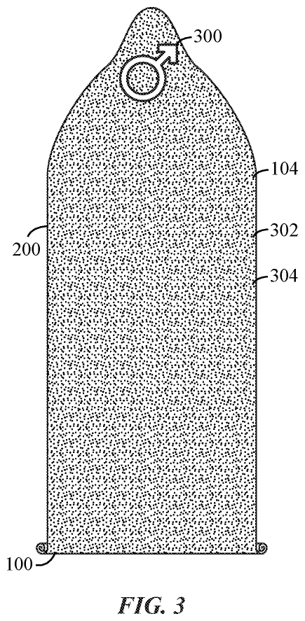

FIG. 3 illustrates a cross-section view of the condom 100 including the tubular body 200 to illustrate the inner surface 104 having a second indicator 300 provided thereon. The inner surface 104 of the tubular body 200 includes a second texture 302 provided on at least a portion of the inner surface 104. In the illustrated example, the second indicator 300 is a male symbol indicating that the second indicator 300 is positioned on the inner surface, which contacts the male during use. The inner surface 104 may include a second color 304, which may be used to further indicate the appropriate orientation of the condom 100 when it is applied.

In some embodiments, the second indicator 300 is positioned at the top end 112 of the inner surface 104 of the condom 100. The second indicator 300 may be various symbols to indicate the surface of the condom 100 on which it is positioned.

The first and second indicators 116,300 may be provided on various positions along the length of the tubular body 200; however, the first and second indicators 116,300 may be positioned at the top end 112 to allow the user to view either indicator when the condom 100 is in a rolled configuration. One skilled in the arts will readily understand that the condom 100 may be provided with only one indicator, such as the first indicator 116.

One skilled in the arts will readily understand that various forms and configurations of indicators may be used, including various symbols to indicate which surface the indicator is placed on to instruct the proper use of the condom 100.

In some embodiments, the first texture 202 and second texture 302 are provided to stimulate the female and/or male users of the condom 100. Similarly, the textures may be used to indicate the correct orientation and application of the condom 100.

During use, the user unwraps the condom 100 from the packaging it is provided within. The user may then examine the condom 100 while it is configured as a roll 102 and determine the correct orientation of the condom 100 using the indicia provided thereon. In some embodiments, the wrapper may include instructions to indicate which surfaces of the condom include specific indicia. For example, the wrapper may indicate the outer surface 114 being the color red and having a ribbed texture. An indicator symbol may be placed on the outer surface 114 to further indicate the correct orientation of the condom.

In some embodiments, the first color 204 and/or second color 304 may be fluorescent or phosphorescent to permit the user to view the condom and colors thereon in a low-light environment. For example, the outer surface 114 or inner surface 104 may be provided with a phosphorescent coloring to glow in the dark and instruct the user how to properly apply and use the condom 100.

In some embodiments, the condom 100 may be provided with a lubricant, spermicide, or combination thereof as is known in the arts.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A condom, comprising:
a tubular body having an open end and a closed end, the tubular body comprising an inner surface and an outer surface, and the tubular body provided in a rolled configuration;
a first texture provided on the inner surface, the first texture providing a first indication of the orientation of the tubular body;
a second texture provided on the outer surface; the second texture providing a second indication of the orientation of the tubular body; and
at least one indicator provided on the inner surface or the outer surface, the indicator providing an indication of the orientation of the tubular body.

2. The condom of claim 1, further comprising a first color provided on the inner surface.

3. The condom of claim 2, further comprising a second color provided on the outer surface.

4. The condom of claim 1, wherein the indicator is a symbol oriented to indicate the closed end of the condom.

5. The condom of claim 1, wherein the indicator is a symbol oriented to indicate the open end of the condom.

6. The condom of claim 1, wherein the second color is visible as an annular ring at a bottom end of the condom when provided in the rolled configuration.

7. A condom, comprising:
a tubular body having an open end and a closed end, the closed end provided on a top end of the condom, the tubular body comprising an inner surface and an outer surface, and the tubular body provided in a rolled configuration;
a first texture provided on the inner surface, the first texture providing a first indication of the orientation of the tubular body;
a second texture provided on the outer surface; the second texture providing a second indication of the orientation of the tubular body; and
at least one indicator provided on the outer surface at the top end of the tubular body, the indicator providing an indication of the orientation of the tubular body, wherein the user utilizes the at least one indicator to properly position the condom during use.

8. The condom of claim 7, further comprising a first color provided on the inner surface.

9. The condom of claim 8, further comprising a second color provided on the outer surface.

10. The condom of claim 9, wherein the indicator is a symbol oriented to indicate the closed end of the condom.

11. The condom of claim 10, wherein the indicator is a symbol oriented to indicate the open end of the condom.

12. The condom of claim 11, wherein the second color is visible as an annular ring at a bottom end of the condom when provided in the rolled configuration.

13. A condom, comprising:
a tubular body extending between an open end and a closed end, the closed end provided on a top end of the condom, the tubular body comprising an inner surface and an outer surface, and the tubular body provided in a rolled configuration wherein the top end is at least partially unrolled;
a first texture provided on the inner surface, the first texture providing a first indication of the orientation of the tubular body, the first texture to stimulate the male user;
a second texture provided on the outer surface; the second texture providing a second indication of the orientation of the tubular body, the second texture to stimulate the female user; and
at least one indicator provided on the outer surface at the top end of the tubular body, the indicator providing an indication of the orientation of the tubular body, wherein the user unwraps the condom and utilizes the at least one indicator to properly position the condom during use.

14. The condom of claim 13, wherein the outer surface is fluorescent.

15. The condom of claim 14, wherein the inner surface is fluorescent.

16. The condom of claim 15, further comprising a first color provided on the inner surface.

17. The condom of claim 16, further comprising a second color provided on the outer surface.

18. The condom of claim 17, wherein the indicator is a symbol oriented to indicate the closed end of the condom.

19. The condom of claim 18, wherein the indicator is a symbol oriented to indicate the open end of the condom.

20. The condom of claim 19, wherein the second color is visible as an annular ring at a bottom end of the condom when provided in the rolled configuration.
